SEQUENCE LISTING (6 sequences)

SEQ ID NO: 1 - protein, 26 aa, Artificial Sequence
  Description of Artificial Sequence Synthetic CD4 peptide
NLHKCQLRCSSLGLLGRCAGSXCACV

SEQ ID NO: 2 - protein, 26 aa, Artificial Sequence
  Description of Artificial Sequence Synthetic CD4 peptide
NLHKCQLRCSSLGLLGRCAGSXCACV

SEQ ID NO: 3 - protein, 27 aa, Artificial Sequence
  Description of Artificial Sequence Synthetic GPR1 peptide
MEDLEETLFEEFENYSYDLDYYSLESC

SEQ ID NO: 4 - protein, 27 aa, Artificial Sequence
  Description of Artificial Sequence Synthetic GPR1 peptide
MEDLEETLFEEFENYSYDLDYYSLESC

SEQ ID NO: 5 - protein, 33 aa, Artificial Sequence
  Description of Artificial Sequence Synthetic polypeptide
XXXXXXXKCXXXXCXXXXXXXXXXCXXXXCXCX

SEQ ID NO: 6 - protein, 32 aa, Artificial Sequence
  Description of Artificial Sequence Synthetic polypeptide
XXXXXXKCXXXXCXXXXXXXXXXCXXXXCXCX

(19) United States
(12) Patent Application Publication
(10) Pub. No.: US 2011/0015368 A1
(43) Pub. Date: Jan. 20, 2011

(54) CD4-RECEPTOR-DERIVED PEPTIDES AND METHOD FOR THE PREPARATION THEREOF

(76) Inventors: Françoise Baleux, Paris (FR); David Bonnaffe, Paris (FR); Hugues Lortat-Jacob, Saint Ismier (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/375,545

(22) PCT Filed: Aug. 3, 2007
(86) PCT No.: PCT/EP07/58069

§ 371 Date: Dec. 8, 2009

(30) Foreign Application Priority Data
Aug. 4, 2006 (FR) 0607149

(51) Int. Cl. C07K 14/00; C07K 1/113

(52) U.S. Cl. 530/324; 530/345

(57) ABSTRACT

The present invention relates to an activated peptide derived from the CD4 receptor, which is capable of coupling by covalent bonding to an organic molecule and thus makes it possible to generate multiple potentially antiviral derivatives. The present invention also relates to a conjugated molecule comprising the CD4-receptor-derived peptide and an organic molecule, preferably the GPR1 peptide or a polyanion. Such a conjugated molecule can in particular be used in antiviral treatments, in particular the treatment of AIDS. The invention also relates to the methods for preparing the activated peptide derived from the CD4 receptor and the conjugated molecule.

This invention relates to an activated peptide derived from the CD4 receptor which is capable, through covalent binding, of coupling to an organic molecule and thus generating numerous potential antiviral derivatives. This invention relates also to a conjugated molecule comprising the CD4-receptor derived peptide and an organic molecule, preferably the peptide GPR1 or a polyanion. Such a conjugated molecule can be used in antiviral treatments, in particular in the treatment of AIDS. This invention further relates to processes for the preparation of the activated peptide derived from the CD4 receptor and of the conjugated molecule.

Triple therapies combining nucleoside (NRTI), non-nucleoside (NNRTI) and/or protease inhibitors (PI) result in a reduction in viral charge beneath levels of detection in a large number of seropositive HIV patients. This efficacy has led to a substantial decrease in the number of deaths resulting from HIV infection. Unfortunately, genotypes with antiviral resistance have been found in 80% of patients and, more worryingly, 45.5% of viral populations are resistant to NRTI/PI combinations while 26% are resistant to a combination of three anti-HIV classes (Tamalet et al., AIDS. 2003 Nov. 7; 17(16):2383-8). This observation is particularly disturbing since the adverse effects of long-term triple therapy treatment (lipoatrophy, lipodystrophy, hypertriglyceridaemia, hypercholesterolaemia, neuropathy, etc.) found in 70% of patients receiving the treatment result in poor compliance and “sudden” discontinuation of treatment which often leads to resistance. The development of less severe forms of treatment with fewer adverse effects and without cross-resistance is therefore a priority despite the large number of currently available medications on the market. With this in mind, it is essential to target HIV replication steps other than reverse transcription and proteolysis.

The discovery of CXCR4 and CCR5 co-receptors (Broder C C & Collman R G, J Leukoc Biol. 1997 July; 62(1):20-9. Review) paved the way for an understanding of the mechanisms of host cell infection. The first step involves attachment of HIV to the cell surface through an interaction between HIV glycoprotein gp120 and the CD4 receptor of the target cell. This is followed by a conformational change in gp120 which exposes an epitope, called CD4i (for induced CD4), previously masked and part of the binding site for co-receptors. The gp120/co-receptor interaction triggers another conformational change leading to exposure of gp41 and this initiates the membrane fusion process. Elucidation of this mechanism made it possible to develop new types of medication which inhibit either the gp120/CD4 interaction (Bristol-Myers Squibb BMS-488043, clinical phase IIa) or the gp120/CCR5 interaction (Schering-Plough SCH-D and Pfizer UK-427,657 at phase III in 2004-05 and GlaxoSmithKline GSK GW873140/AK602 at phase I) or the fusion phase by binding to gp41 (T20, Fuzeon®, Roche).

These extremely positive results demonstrate that approaches aimed at blocking one of the stages of HIV penetration into the cell are relevant. Nevertheless, it is to be noted that among inhibitors of the gp120/co-receptor interaction currently being developed clinically, only products which inhibit the interaction with CCR5 have been studied. The development of compounds which bind to CXCR4 has been stopped, such as AMD8664 which causes cardiovascular adverse effects linked to its mechanism of action (Gao Z & Metz W A, 2003 September; 103(9):3733-52). The absence of treatment which inhibits the gp120/CXCR4 interaction might eventually lead to failure of treatment targeting CCR5 by triggering a change in the viral population towards X4 tropism (infecting HIV via CXCR4), often associated with accelerated depletion of CD4+ T lymphocytes. Curiously, the binding site for co-receptors, highly conserved in HIV-1, HIV-2 and SIV (Rizzuto C D et al., 1998 Jun. 19; 280(5371):1949-53; Kwong P D et al., Nature. Jun. 18; 393(6686):648-59), does not appear to be a therapeutic target on which much research is conducted. The fact that this site is hidden as long as gp120 is not bound to CD4 makes molecular access to this site difficult. This invention proposes a solution to this problem by the preparation, via chemical synthesis, of compounds that are capable of binding to the preserved binding site for co-receptor as well as to the V3 loop. Such compounds should be in principle capable of inhibiting gp120/CD4 interactions and gp120/co-receptor interactions and this whatever the HIV strain.

It has been known for many years that certain polyanions such as heparin (HP) and dextran sulphate (DS) but not chondroitin sulphate (CS) are capable of inhibiting the infection of cells by HIV (Esté J A et al., Mol. Pharmacol. 1997 July; 52(1):98-104). They are nonetheless not used clinically, particularly as a result of their anticoagulant effects (Flexner C et al., Antimicrob Agents Chemother. 1991 December; 35(12):2544-50). It has recently been shown that the molecular mechanism of this inhibition is linked to interaction of the polyanion with the V3 loop (Moulard M et al., J. Virol. 2000 February; 74(4):1948-60).

Moreover, various studies have explored the use of soluble CD4 to inhibit interaction of the virus with CD4 expressed at the surface of HIV target cells. This solution was found to be ineffective since soluble CD4, by binding to the virus, exposes the epitope CD4i and thus encourages interaction of the virus with the CCR5 or CXCR4 co-receptor which, in some cases, increases infection (Schenten D. et al., 1999. J. Virol. 73:5373-80).

It is known from international patent application WO 03/089000 that a peptide derived from the CD4 receptor, when contacted with a polyanion, has an anti-HIV activity. In particular, it is recommended that compounds in which the peptide and polyanion are bound can be prepared according to the description given in the article by Najjam S. et al. (Cytokine 1997, 9 (12):1013-1022) (refer to point I.1 in the EXAMPLES section).

In this invention, the inventors have obtained activated peptides derived from the CD4 receptor likely to bind directly and covalently to the polyanion or any other organic molecule likely to play a role in anti-HIV activity with miniCD4. This activation requires the insertion of specific amino acid residues into the native peptide. In particular, the inventors have discovered that the presence of one and only one amino acid lysine residue in the sequence of the peptide derived from the CD4 receptor is vital to obtaining an activated peptide according to the invention. Moreover, this sole amino acid lysine residue has to be in a well-defined position in the sequence of the peptide derived from the CD4 receptor. Devising a miniCD4 peptide containing a single amino acid lysine residue in a defined position makes it possible to introduce the desired function selectively and directly onto miniCD4.

This invention therefore offers activated compounds which make it possible to produce numerous potential antiviral derivatives. These derivatives consist of conjugated molecules comprising a CD4 peptide specifically coupled to an organic molecule such as a polyanion by means of a linker.

This approach is therapeutically advantageous to inhibit viral attachment to cells as it directly targets the virus and not the cells themselves. It is therefore, at first sight, devoid of the cellular effects observed with medication which binds to co-receptors. In addition, in view of the preservation of the sites involved as a function of various viral tropisms, the compounds according to the invention should interact with the gp120 of different viral isolates. Also, while it might be misleading to think that resistance will not occur, this new type of compound should not lead to easy emergence of resistance. Indeed, the CD4 site of gp120 has to remain intact in order to continue to bind to CD4, as do the basic residues involved in binding to the polyanion for interaction with the co-receptors. Mutation in one of these two sites would result in a virus that is not very functional. Finally, within the framework of the invention, a wholly synthetic version of the compounds can be developed, thus guaranteeing a preparation available in large quantities and one that is homogeneous and perfectly defined. The coupling method is simple, rapid and quantitative.

Therefore, according to a first aspect, the invention relates to a process for the preparation of an activated peptide derived from the CD4 receptor, said peptide, once activated, being capable of coupling to an organic molecule by means of a covalent bond and wherein said peptide derived from the CD4 receptor comprises or consists of the following general sequence (I):

Xaa^{f} - P1 - Lys - Cys - P2 - Cys - P3 - Cys -
Xaa^{g} - Xaa^{h} - Xaaⁱ - Xaa^{j} - Cys - Xaa^{k} - Cys -
Xaa^{l} - Xaa^{m}, (I)

in which:

P1 represents 3 to 6 amino acid residues,

P2 represents 2 to 4 amino acid residues,

P3 represents 6 to 10 amino acid residues,

Xaa^{f} represents N-acetylcysteine (Ac-Cys) or thiopropionic acid (TPA),

Xaa^{g} represents Ala or Gln,

Xaa^{h} represents Gly or (D)Asp or Ser,

Xaaⁱ represents Ser or His or Asn

Xaa^{j} represents biphenylalanine (Bip), phenylalanine or [beta]-naphthylalanine,

Xaa^{k} represents Thr or Ala, and

Xaa^{l} represents Gly, Val or Leu,

Xaa^{m} represents —NH₂ or —OH,

the amino acid residues in P1, P2 and P3 being natural or non-natural, identical or different, said residues of P1, P2 and P3 all being different from the Lys residue and P1, P2 and P3 having a sequence in common or not,
characterized in that the process involves contacting the peptide of general sequence (I) derived from the CD4 receptor with a bifunctional compound carrying two active groups, where at least one of the two active groups is capable of forming a covalent bond with free amino group (—NH2) of the amino acid Lys residue present in general sequence (I).

Preferably, P3 comprises at least one basic amino acid, said basic amino acid being even more preferably arginine. The presence of basic residues in this portion of the CD4 receptor fragment contributes to its binding to the gp120 protein. The inventors therefore prefer to introduce at least one basic amino acid into P3, preferably arginine. This maintains thus a basic charge which is not reactive at derivation at pH 7-8 but which has been found to be useful for the binding of miniCD4 peptide to the gp120 protein.

In this application, the terms “miniCD4 peptide”, “CD4 peptide” and “miniCD4” are used interchangeably to designate the peptide derived from the CD4 receptor comprising or consisting of general sequence (I) defined above.

This invention needs the peptide derived from the CD4 receptor to include in its general sequence (I) one and only one amino acid lysine residue (Lys) in the position defined in general sequence (I).

The Cys residues in general sequence (I) allow the formation of three disulphide bridges needed for folding back of miniCD4.

Thiopropionic acid (TPA), when it is in the N-terminus position of the peptide of general sequence (I), makes it possible to reduce hindrance in N-ter and overcome the presence of an amine group.

Thus, according to a preferred embodiment, Xaa^{f} represents TPA in general sequence (I).

In general sequence (I), Xaa^{j} represents Bip, Phe or [beta]-naphthylalanine. Biphenylalanine increases contact with glycoprotein gp120 in the cavity where the Phe 43 of CD4 receptor is lodged. Nevertheless, a miniCD4 peptide according to the invention with a Phe may mimic CD4 better when the structure of the miniCD4/gp120 complex is analyzed (Huang C C et al., Structure. 2005 May; 13(5):755-68).

Thus according to another preferred embodiment, Xaa^{j} represents Phe.

The peptide of general sequence (I) derived from the CD4 receptor has an alpha helix structure followed by a beta sheet. The amino acids Xaa^{g}-Xaa^{h}-Xaaⁱ-Xaa^{j}-Cys-Xaa^{h}-Cys-Xaa^{l} participate in a major way to the binding to gp120. These peptides have CI₅₀ (affinity for gp120) similar to those of sCD4 (soluble CD4).

The peptide of general sequence (I) derived from the CD4 receptor can be prepared by conventional solid phase chemical synthesis techniques, for example according to the Fmoc solid phase peptide synthesis method (“Fmoc solid phase peptide synthesis, a practical approach”, edited by W. C. Chan and P. D. White, Oxford University Press, 2000) and/or by genetic recombination.

Preferably, the sequence of the peptide derived from the CD4 receptor of general sequence (I) is chosen from the group consisting of sequences SEQ ID No. 1 and SEQ ID No. 2, advantageously SEQ ID No. 1.

The term “bifunctional compound” in this patent application refers to any compound incorporating two active groups where at least one of the two groups is capable of forming a covalent bond with the free amino group (—NH2) of the amino acid Lys residue present in general sequence (I).

The person skilled in the art knows well the bifunctional compounds which can be used within the framework of this invention. Namely, the bifunctional compound according to this invention can be chosen from the following non-limiting list: NHS-PEOₙ-Maleimide where n is comprised between 2 and 24, advantageously n=2, 4, 8 or 12, SMPT (4-succinimidyloxycarbonyl-methyl-a-[2-pyridyldithio]toluene), Sulfo-LC-SMPT (4-sulfosuccinimidyl-6-methyl-a-(2-pyridyldithio)toluamido]hexanoate)), Sulfo-KMUS (N-[k-maleimidoundecanoyloxy]sulfosuccinimide ester), LC-SMCC (succinimidyl-4-[N-maleimidomethyl]cyclohexane-1-carboxy-[6-amidocaproate]), KMUA (N-k-maleimidoundecanoic acid), Sulfo-LC-SPDP (sulfosuccinimidyl 6-(3′-[2-pyridyldithio]-propionamido)hexanoate), LC-SPDP (succinimidyl 6-(3-[2-pyridyldithio]-propionamido)hexanoate), SMPB(succinimidyl 4-[p-maleimidophenyl]butyrate), Sulfo-SMP (sulfosuccinimidyl 4-[p-maleimidophenyl]butyrate), Sulfo-SIAB (N-sulfosuccinimidyl[4-iodoacetyl]aminobenzoate), SIAB (N-succinimidyl[4-iodoacetyl]aminobenzoate), Sulfo-EMCS ([N-e-maleimidocaproyloxy]sulfosuccinimide ester), EMCA (N-e-maleimidocaproic acid), EMCS ([N-e-maleimidocaproyloxy]succinimide ester), SMCC (succinimidyl 4-[N-maleimidomethyl]cyclohexane-1-carboxylate), Sulfo-SMCC (sulfosuccinimidyl 4-[N-maleimidomethyl]cyclohexane-1-carboxylate), MBS (m-maleimidobenzoyl-N-hydroxy succinimide ester), Sulfo-MBS(m-maleimidobenzoyl-N-hydroxysulfosuccinimide ester), GMBS (N-[g-maleimidobutyryloxy]succinimide ester), Sulfo-GMBS (N-[g-maleimidobutyryloxy]sulfosuccinimide ester), SPDP (N-succinimidyl-3-(2-pyridyldithio)propionate), SBAP (succinimidyl 3-[bromoacetamido]propionate), BMPS(N-[[beta]-maleimidopropyloxy]succinimide ester), BMPA (N-[beta]-maleimidopropionic acid), AMAS N-(a-maleimidoacetoxy) succinimide ester), SIA (N-succinimidyl iodoacetate), SMPH(succinimidyl-6-[beta-maleimidopropionamido]hexanoate), SATA (N-succinimidyl-S-acetylthioacetate), SATP (N-succinimidyl-S-acetylthiopropionate).

According to the invention, NHS-PEOₙ-Maleimide where n=2 is also called succinimidyl-[(N-maleimidoproprionamido)-diethyleneglycol]ester, NHS-PEOₙ-Maleimide where n=4 is also called succinimidyl-[(N-maleimidoproprionamido)-tetraethyleneglycol]ester, NHS-PEOₙ-Maleimide where n=8 is also called succinimidyl-[(N-maleimidoproprionamido)-octaethyleneglycol]ester, NHS-PEOₙ-Maleimide where n=12 is also called succinimidyl-[(N-maleimidoproprionamido)-dodecaethyleneglycol]ester.

The active group capable of forming a covalent bond with the free amine group (—NH₂) of the amino acid Lys residue present in general sequence (I) can be any active ester group.

Preferably, the active group capable of forming a covalent bond with the free amine group (—NH₂) of the amino acid Lys residue present in general sequence (I) is the active group N-hydroxysuccinimide ester (NHS).

Even more preferably, the two active groups of the bifunctional compound are different (heterobifunctional group) and one of the two groups is the NHS active group.

According to a particularly preferred embodiment, the bifunctional compound is succinimidyl-6-[beta-maleimidoproprionamido]hexanoate (SMPH).

The molecular structure of SMPH is as follows:

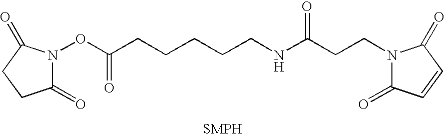

According to another particularly preferred embodiment, the bifunctional compound is chosen from the group consisting of N-succinimidyl-S-acetylthioacetate (SATA) and N-succinimidyl-S-acetylthiopropionate (SATP).

The molecular structure of SATA is as follows:

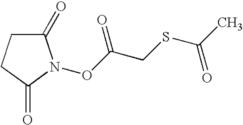

The molecular structure of SATP is as follows:

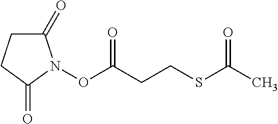

According to yet another preferred embodiment, the bifunctional compound is succinimidyl-[(N-maleimidopropionamido)-diethyleneglycol]ester, also called NHS-PEO₂-maleimide, succinimidyl-[(N-maleimidopropionamido)-tetraethyleneglycol]ester, also called NHS-PEO₄-maleimide, succinimidyl-[(N-maleimidopropionamido)-octaethyleneglycol]ester, also called NHS-PEO₈-maleimide, succinimidyl-[(N-maleimidopropionamido)-dodecaethyleneglycol]ester, also called NHS-PEO₁₂-maleimide, still more preferably the bifunctional compound is NHS-PEO₂-maleimide.

The molecular structure of NHS-PEO₂-maleimide is as follows:

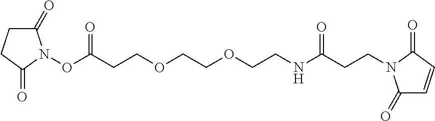

The bifunctional compounds can be obtained from PIERCE (Rockford, Ill.).

Preferably again, the process for preparation of an activated peptide according to the invention includes a preliminary stage for the preparation of the peptide derived from the CD4 receptor of general sequence (I) where Xaa^{f} represents TPA, wherein the peptide derived from the CD4 receptor of general sequence (II) below:

P1-Lys-Cys-P2-Cys-P3-Cys-Xaa^{g}-Xaa^{h}-Xaaⁱ-Xaa^{j}-Cys-Xaa^{k}-Cys-Xaa^{l}-Xaa^{m}, (II)

wherein P1 to P3, Bip and Xaa^{g} to Xaa^{m} are as defined in general sequence (I) above,
is contacted with N-succinimidyl-3-(2-pyridyldithio)propionate (SPDP) in order to incorporate TPA into the N-terminus of said peptide derived from the CD4 receptor of general sequence (II).

The molecular structure of SPDP is as follows:

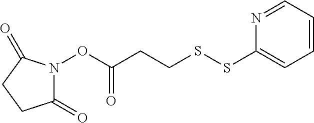

According to a second aspect, this invention relates to an activated peptide derived from the CD4 receptor comprising or consisting of general sequence (I) as defined above wherein the amino acid Lys residue is covalently bound, advantageously by an amine bond, to an active group capable of coupling to an organic molecule by means of a covalent bond.

As examples of active groups capable of coupling to an organic molecule by means of a covalent bond, the following groups can be cited: maleimide, bromoacetyl, S-S-pyridinium. When miniCD4 is activated by a protected thiol group (e.g. thioacetyl), it is possible to carry out coupling to an organic molecule which carries a maleimide group for example. This is possible when functionalisation of a polysaccharide (or polyanion) by a thiol group or thioacetyl group poses a problem. This then called “reverse coupling”.

Preferably, the active group is the maleimide group.

The molecular structure of the activated peptide according to the invention whose active group is maleimide is the following when SMPH is the bifunctional compound used:

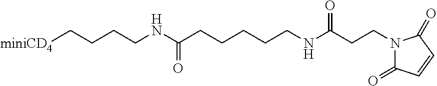

In this application, the term “SMPH activated miniCD4 peptide” refers to an activated peptide according to the invention whose amino acid Lys residue is covalently bound, advantageously by an amine bond, to a maleimide active group via a linker derived from SMPH.

According to another advantageous embodiment, the molecular structure of the activated peptide according to the invention whose active group is the maleimide group is the following when NHS-PEO₂-maleimide is the bifunctional compound used:

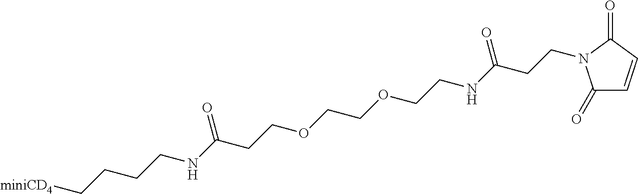

In this application, the term “maleimide activated miniCD4 peptide via a PEO₂ linker” refers to an activated peptide according to the invention whose amino acid Lys residue is covalently bound, advantageously by an amine bond, to a maleimide active group via a PEO₂ linker.

According to another preference, the active group is the thioacetyl group.

For example, the molecular structure of the activated peptide according to this invention whose active group is the thioacetyl group is the following when SATA is the bifunctional compound used:

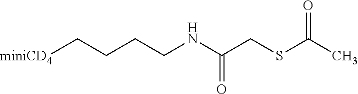

Similarly, the molecular structure of the activated peptide according to this invention whose active group is the thioacetyl group is the following when SATP is the bifunctional compound used:

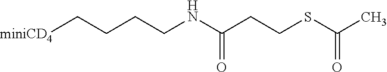

The thioacetyl group is a protected form of the thiol group. To deprotect the thiol group, we use hydroxylamine for example. This step is carried out simultaneously to coupling to the maleimide group carried by the organic molecule.

In this application, the terms “SATA activated miniCD4 peptide” and “SATP activated miniCD4 peptide” refer to an activated peptide according to the invention whose amino acid Lys residue is covalently bound, advantageously by an amine bond, to a protected thiol group (e.g. thioacetyl) via a linker derived from SATA or SATP.

According to a third aspect, the invention relates to a process for the preparation of a conjugated molecule comprising a peptide derived from the CD4 receptor coupled by covalent binding to an organic molecule, said peptide derived from the CD4 receptor comprising or consisting of general sequence (I) as defined above wherein the process involves contacting the activated peptide according to the invention as defined above with the organic molecule. The activated peptide derived from the CD4 receptor comprises general sequence (I) as defined above wherein the amino acid Lys residue is covalently bound, advantageously by an amine bond, to an active group capable of coupling to an organic molecule by means of a covalent bond.

According to a particular embodiment, the active group is the maleimide group and the organic molecule carries a thiol or thioacetyl group.

Preferably, the organic molecule carrying a thiol group is the peptide GPR1 whose sequence is chosen from the group consisting of sequences SEQ ID No. 3 and SEQ ID No. 4, advantageously SEQ ID No. 3.

Peptide GPR1 of sequence SEQ ID No. 3 or SEQ ID No. 4 is a synthetic peptide derived from the extracellular N-terminus region of the receptor coupled to protein G, GPR1 (Jinno-Oue et al., J Biol chem. 2005 Sep. 2; 280(35):30924-34. Epub 2005 May 26).

According to another preference, the organic molecule carrying a thiol group is chosen from the group consisting of peptides essentially carrying acid residues (aspartic acid, glutamic acid, etc.), peptides essentially carrying phosphorylable residues (Ser, Thr, Tyr, etc.) and peptides essentially carrying sulphatable residues (Ser, Thr, Tyr, etc.).

According to another preference, the organic molecule carrying a thiol group is a modified polyanion carrying a thiol or thioacetyl group.

According to the invention, the polyanion or polysaccharide can also be advantageously chosen from the group consisting of heparin, heparan sulphate and a polyanion equivalent to heparin and heparan sulphate. For example, this includes dextran sulphate (brand by UENO FINE CHEMICALS), curdlan sulphate (brand by AJINOMOTO), naphthalene-2 sulfonate polymer (brand by PROCEPT), pentosan polysulphate (brand by BAKER NORTON PHARM; HOESCHST) or resobene (brand).

It is preferable that the polyanion is not too long as it would have anticoagulant activity, not desirable in this invention, and would form aspecific bonds with various proteins, namely thrombin and antithrombin III. Its length is preferably similar to that of a heparin chain with a degree of polymerisation such as defined below. The polyanion has preferably at least two anionic groups per disaccharide.

According to this invention, when the polyanion is heparin or heparan sulphate, it has preferably a degree of polymerisation dp of 10 to 24, advantageously 12 to 24, preferably 16 to 22. According to the invention, heparin, heparan sulphate or the polyanion equivalent to heparin or heparan sulphate can have a degree of polymerisation dp of 12 to 20, for example 15 to 17.

Heparin dodecasaccharide (HP₁₂) can be cited as an example.

According to a particular embodiment, the modified polyanion carrying the thiol or thioacetyl group is chosen from the group consisting of heparin and heparane sulphate and has a degree of polymerisation dp of 10 to 24.

According to another particular embodiment, the active group is the thioacetyl group and the organic molecule carries a maleimide or halogen group.

According to the invention, the polyanion can be prepared by partial depolymerisation of heparin or heparane sulphate using an enzymatic method, for example by means of heparinase, or chemical method, for example by means of nitrous acid. When they are obtained chemically, the heparans can be defined by the presence of N-sulphated or N-acetylated glucosamine or nonsubstituted in position N bound to uronic acid (glucuronic acid or iduronic acid) with a varying proportion of sulphate. Structural analogues of these oligosaccharides can be obtained by chemical synthesis.

There are many advantages of such a synthetic approach compared to conjugation of recombinant compounds or those from natural sources. Within the framework of therapeutic usage, synthetic compounds are always preferable as, in addition to a fully defined structure, contamination by pathogens can be avoided, especially prion proteins in the case of HP fragments. Moreover, synthetic HP fragments are much more homogeneous than their natural equivalents. For example, synthetic HP₁₂ is totally devoid of 3-O-sulphate groups which are responsible for heparin's antithrombin activity.

The operating conditions for the processes according to the invention for preparation of the activated peptide and conjugated molecule are well known to the person skilled in the art and can be modified if necessary.

According to a fourth aspect, this invention relates to a conjugated molecule comprising a peptide derived from the CD4 receptor comprising or consisting of general sequence (I) as defined above coupled to an organic molecule wherein the peptide derived from the CD4 receptor and the organic molecule are coupled to each other with a linker and wherein the amino acid Lys residue of general sequence (I) forms an amino bond with the linker.

This invention also relates to a conjugated molecule comprising a peptide derived from the CD4 receptor comprising or consisting of general sequence (I) as defined above coupled to an organic molecule, said conjugated molecule being likely to be obtained by the process according to the invention for the preparation of a conjugated molecule.

In this application, the term “linker” refers to any agent which binds the miniCD4 peptide of the invention to the organic molecule, said linker varying as a function of the bifunctional compound used.

It is preferably a conjugated molecule according to the invention wherein the sequence of the peptide derived from the CD4 receptor comprises or consists of general sequence (I), preferably sequence SEQ ID No. 1, and the organic molecule carrying a thiol group is the peptide GPR1 whose sequence is chosen from the group consisting of sequences SEQ ID No. 3 and SEQ ID No. 4, advantageously SEQ ID No. 3.

Coupling according to the invention of peptide GPR1 to the peptide derived from the CD4 receptor of general sequence (I) is capable of attaching to HIV glycoprotein gp120 and concomitantly blocking attachment of the virus to CD4 and to the CXCR4 and CCR5 co-receptors.

The molecular structure of such a conjugated molecule, including a peptide derived from the CD4 receptor of general sequence (I) coupled to peptide GPR1 of sequence SEQ ID No. 3 and SEQ ID No. 4, is as follows when SMPH was used for the coupling:

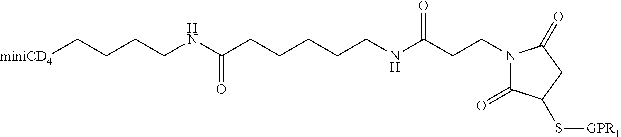

According to another preference, this is a conjugated molecule according to the invention wherein the sequence of the peptide derived from the CD4 receptor comprises or consists of general sequence (I), preferably sequence SEQ ID No. 1, and the organic molecule carrying a thiol group is the modified polyanion carrying the thiol or thioacetyl group.

The molecular structure of such a conjugated molecule, including a peptide derived from the CD4 receptor of general sequence (I) coupled to a modified polyanion carrying the thiol or thioacetyl group, is as follows when SMPH was used for the coupling:

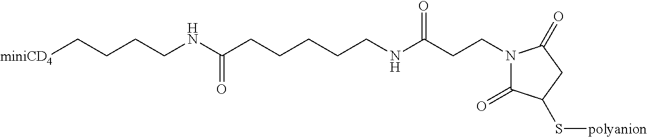

It can also be the following conjugated molecule when NHS-PEO₂-maleimide is the bifunctional compound used:

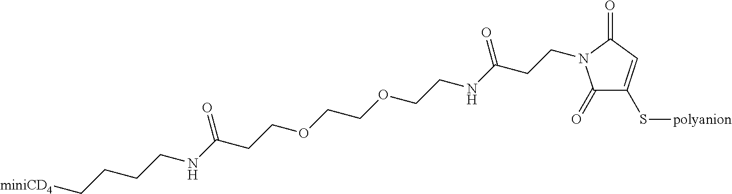

Preferably, the modifier polyanion carrying the thiol or thioacetyl group is chosen from among the group consisting of heparin and heparan sulphate and the degree of polymerisation dp is from 10 to 24.

According to another embodiment, the conjugated molecule according to the invention comprises a peptide derived from the CD4 receptor comprising or consisting of general sequence (I), preferably sequence SEQ ID No. 1, and an organic molecule carrying a maleimide or halogen group.

For example, the molecular structure of such a conjugated molecule including a peptide derived from the CD4 receptor of general sequence (I) coupled to an organic molecule carrying a maleimide group is as follows when SATA is used for the coupling:

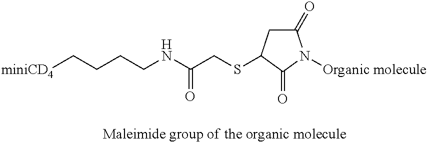

It results from the invention that the conjugated molecule as defined above includes a linker whose length varies as a function of the bifunctional compounds used.

According to another embodiment, the conjugated molecule according to the invention comprises a peptide derived from the CD4 receptor comprising or consisting of general sequence (I), preferably SEQ ID No. 1, and the organic molecule carrying a thiol group chosen from the group consisting of peptides essentially carrying acid residues (aspartic acid, glutamic acid, etc.), peptides essentially carrying phosphorylable residues (Ser, Thr, Tyr, etc.) and peptides essentially carrying sulphatable residues (Ser, Thr, Tyr, etc.).

This invention also relates to use of the activated peptide derived from the CD4 receptor comprising or consisting of general sequence (I) as defined above wherein the amino acid Lys residue is covalently bound, advantageously by an amine bond, to an active maleimide group, for the coupling to an organic molecule carrying a thiol or thioacetyl group by means of a covalent bond.

This invention further relates to the use of the activated peptide derived from the CD4 receptor comprising or consisting of general sequence (I) as defined above wherein the amino acid Lys residue is covalently bound, advantageously by an amine bond, to a protected active thiol group (e.g. thioacetyl), for thz coupling to an organic molecule carrying a maleimide or halogen group by means of a covalent bond

The invention also relates to the use of the activated peptide derived from the CD4 receptor comprising or consisting of general sequence (I) as defined above wherein the amino acid Lys residue is covalently bound, advantageously by an amine bond, to an active maleimide group allowing the coupling to an organic molecule carrying a thiol or thioacetyl group by means of a covalent bond for the manufacture of a medicament for antiviral treatment.

The invention moreover relates to the use of the activated peptide derived from the CD4 receptor comprising or consisting of general sequence (I) as defined above wherein the amino acid Lys residue is covalently bound, advantageously by an amino bond, to a protected active thiol group (e.g. -thioacetyl) allowing the coupling to an organic molecule carrying a maleimide or halogen group by means of a covalent bond for the manufacture of a medicament for antiviral treatment.

Preferably, the invention relates to the use of the activated peptide derived from the CD4 receptor comprising or consisting of general sequence (I) as defined above wherein the amino acid Lys residue is covalently bound, advantageously by an amine bond, to an active maleimide group allowing the coupling to an organic molecule carrying a thiol or thioacetyl group by means of a covalent bond for the manufacture of a medicament for the treatment of AIDS.

The invention further relates to a conjugate molecule according to the invention for its use as medicament.

According to yet another aspect, the invention relates to the use of a conjugated molecule according to the invention for the manufacture of a medicament for the treatment of AIDS.

The invention also concerns an antiviral treatment method, preferably an anti-AIDS treatment method, comprising the use of a conjugated molecule according to the invention.

Preferably, the conjugated molecule according to this invention is a conjugated molecule in which general sequence (I) is sequence SEQ ID No. 1, and the organic molecule carrying a thiol group is the peptide GPR1 whose sequence is chosen from the group consisting of sequences SEQ ID No. 3 and SEQ ID No. 4, advantageously SEQ ID No. 3.

According to another preference, the conjugated molecule according to this invention is a conjugated molecule wherein general sequence (I) is SEQ ID No. 1, and the organic molecule carrying a thiol group is chosen from the group consists of peptides essentially carrying acid residues (aspartic acid, glutamic acid, etc.), peptides essentially carrying phosphorylable, residues (Ser, Thr, Tyr, etc.) and peptides essentially carrying sulphatable residues (Ser, Thr, Tyr, etc.).

According to another preference, the conjugated molecule according to the invention is a conjugated molecule in which general sequence (I) is sequence SEQ ID No. 1, and the organic molecule carrying a thiol group is the modified polyanion carrying the thiol or thioacetyl group.

The embodiments described above apply to these different aspects of the invention.

The examples and figures below illustrate the invention but do not limit its scope in any way.

### FIGURES

FIG. 1: Diagram of the synthesis of activated peptide SMPH or SATA/SATP derived from the CD4 receptor and of the miniCD4-GPR1 conjugated molecule.

FIG. 2: Final HPLC Elugram of the miniCD4-GPR1 conjugated molecule.

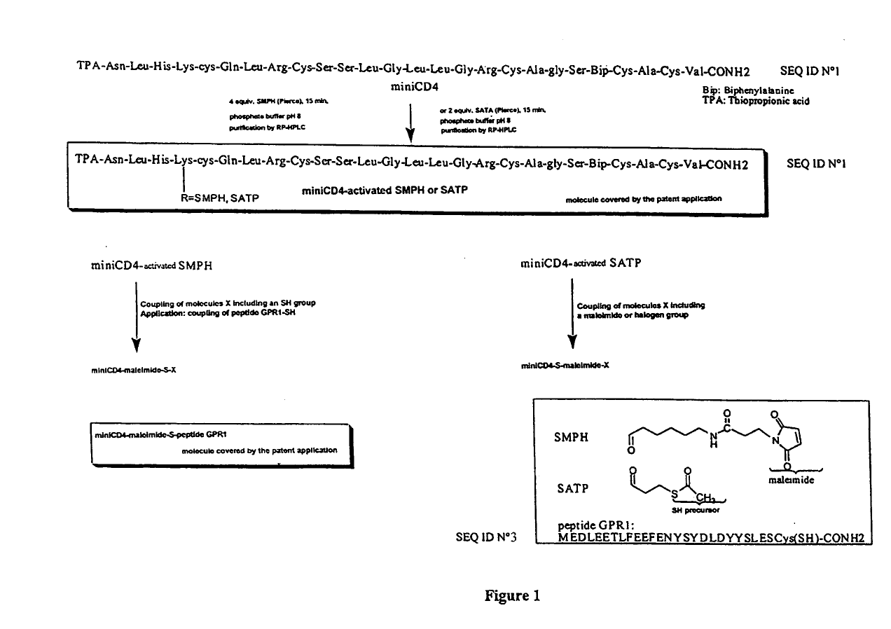

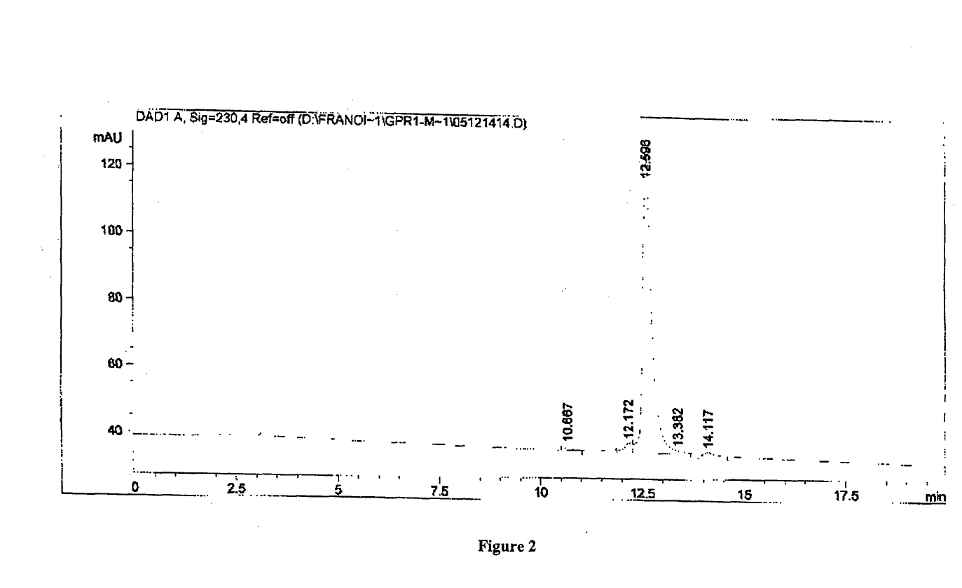

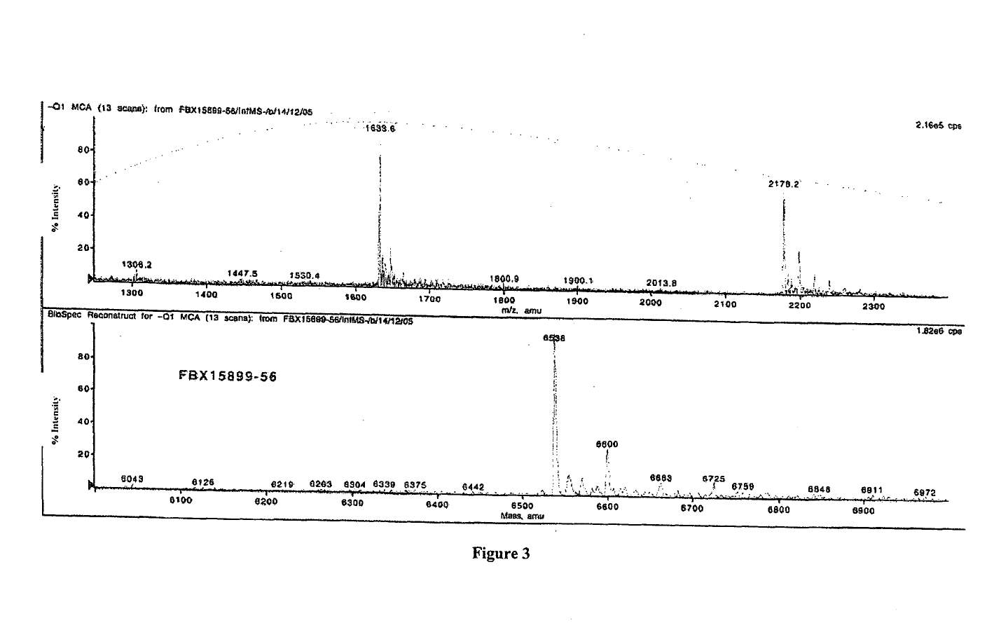

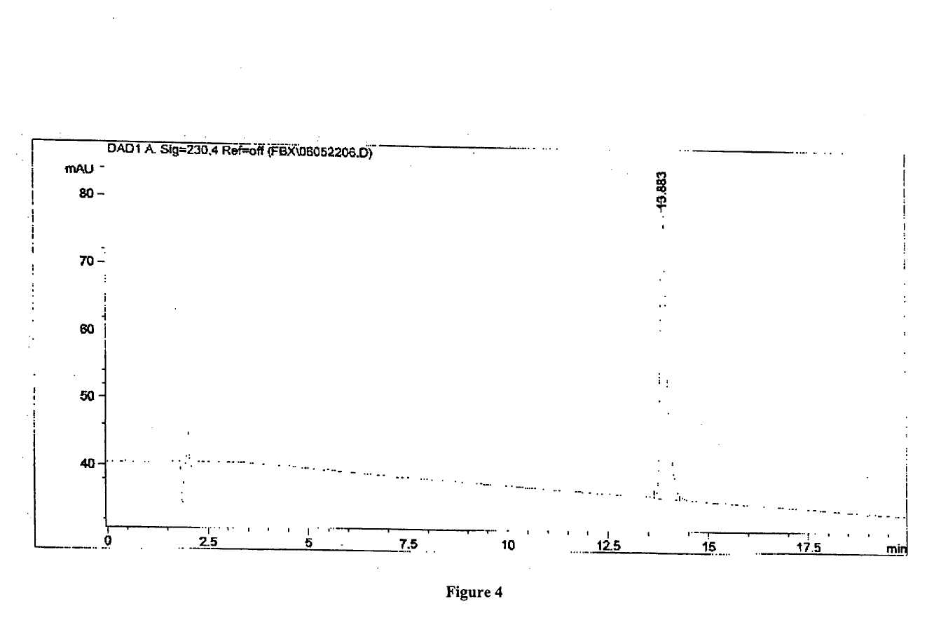

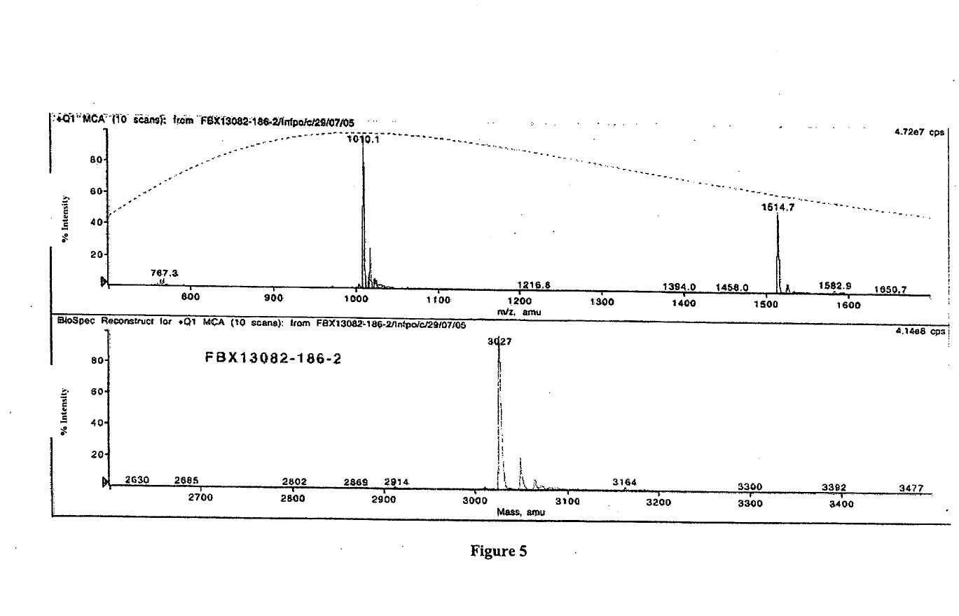

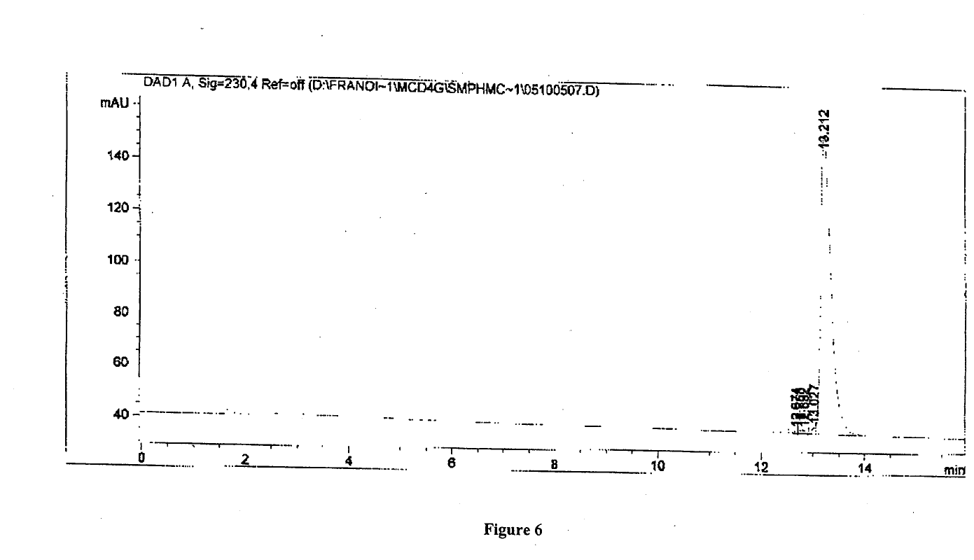

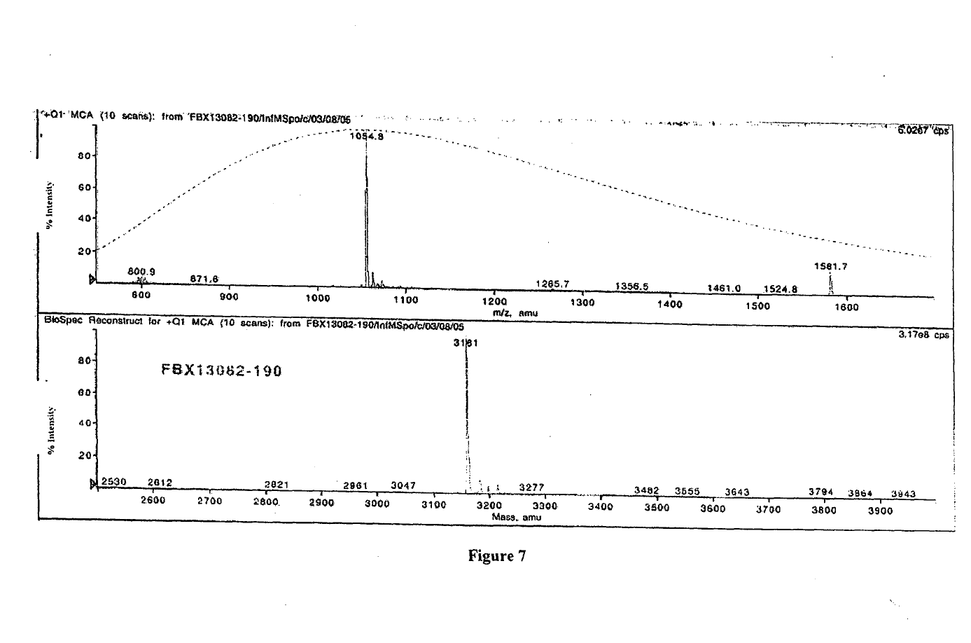

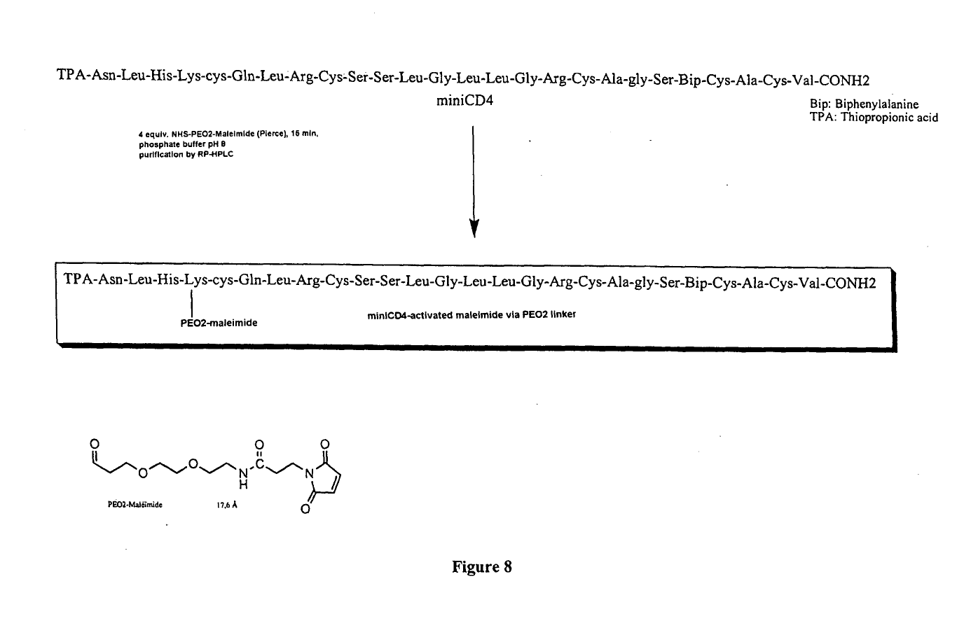

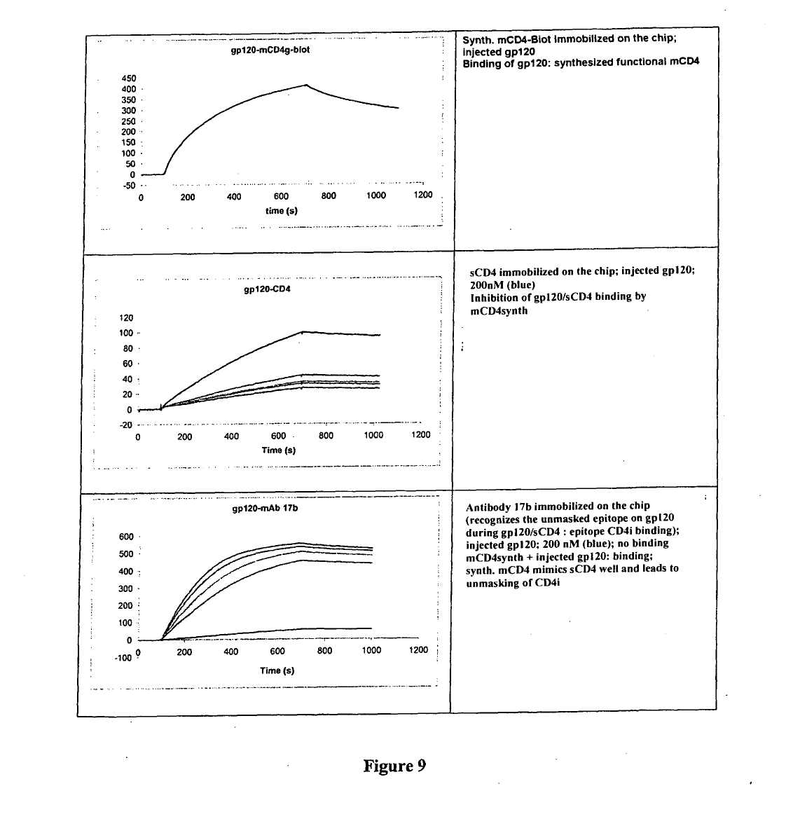

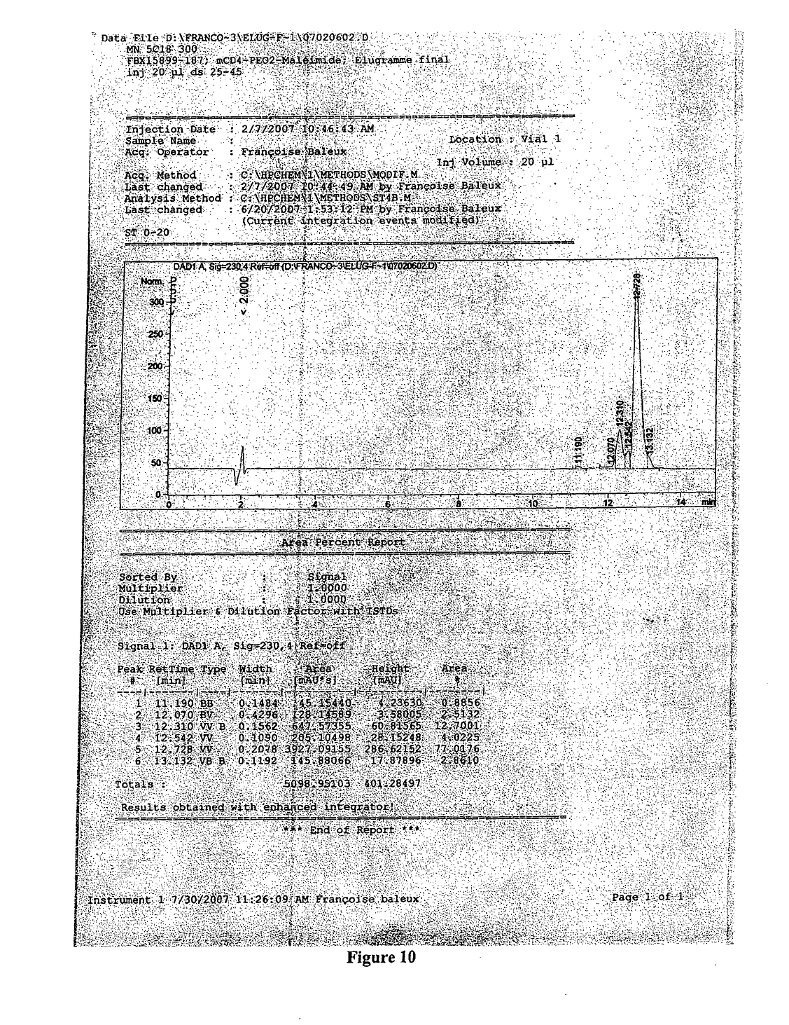

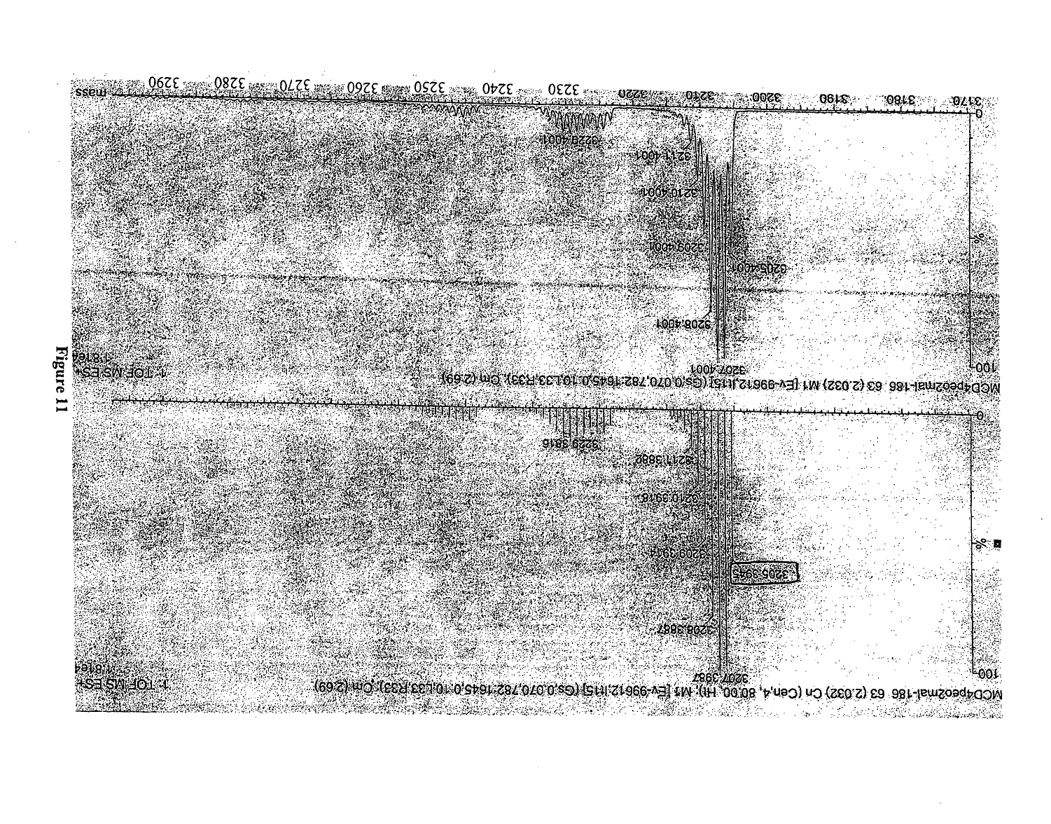

Epsilon ds 50 μl TFA/CH3CN 35%; inj 10 μl ds 35-55 Sample name: GPR1-mCD4

### Surface Percentage Ratios:

Sorted by: signal

### Multiplicator: 1.0000

### Dilution: 1.0000

Uses multiplicator and dilution factor with internal standard

### Signal 1: DADI A, Sig=230.4 Ref=off

TABLE 1

| | Retention | | | Surface | | |
|---|---|---|---|---|---|---|
| Peak | time | | Width | area | Height | Surface |
| no. | [min] | Type | [min] | [mAU * s] | [mAU] | area % |
| 1 | 10.667 | BB | 0.1595 | 14.20322 | 1.29982 | 0.9401 |
| 2 | 12.172 | VV | 0.1905 | 39.30429 | 2.93002 | 2.6016 |
| 3 | 12.596 | VV | 0.2345 | 1422.81519 | 89.94869 | 94.1780 |
| 4 | 13.382 | VV B | 0.2054 | 18.64892 | 1.12492 | 1.2344 |
| 5 | 14.117 | BV | 0.2103 | 15.80048 | 1.02216 | 1.0459 |

| | | |
|---|---|---|
| Totals | 1510.77210 | 96.32562 |

Results obtained with improved integrator.

FIG. 3: Mass spectrum of the miniCD4-GPR1 conjugated molecule.

Copy 3 of the hypermass calculation for −Q1 MCA (13 scans): from FBX15899-56/InfMS-/b/14/12/05
Criteria used for hypermass calculation
Agent:, Mass: 1.0079, Charge: 1, Agent lost
Tolerance for charge estimation: 0.1000
Tolerance between mass estimations: 20.000

TABLE 2

| | | | Calculated | Hypermass |
|---|---|---|---|---|
| Peak | Intensity | Charge | charge | estimation |
| 1306.33 | 10500.00 | 5 | 4.99783 | 6536.67 |
| 1633.34 | 215500.00 | 4 | 3.99783 | 6537.38 |
| 2178.24 | 139000.00 | 3 | 2.99935 | 6537.74 |

Estimated final mass: 6537.26
Standard deviation: 0.54

FIG. 4: Final HPLC Elugram of SATP activated miniCD4 peptide.

25-45 in 20 minutes
Sample name: FBX13082-168-2

### Surface Percentage Ratios:

Sorted by: signal

### Multiplicator: 1.0000

### Dilution: 1.0000

Uses multiplicator and dilution factor with internal standard

### Signal 1: DADI A, Sig=230.4 Ref=off

TABLE 3

| | Retention | | | Surface | | |
|---|---|---|---|---|---|---|
| Peak | time | | Width | area | Height | Surface |
| no. | [min] | Type | [min] | [mAU * s] | [mAU] | area % |
| 1 | 13.883 | VV | 0.1668 | 520.71381 | 46.44330 | 100.0000 |

| | | |
|---|---|---|
| Totals | 520.71381 | 46.44330 |

Results obtained with improved integrator.

FIG. 5: Mass spectrum of SATP activated miniCD4 peptide

Copy of the hypermass calculation for +Q1 MCA (10 scans): from FBX13082-186-2/Infpo/c/29/07/05
Criteria used for hypermass calculation
Agent:, Mass: 1.0079, Charge: 1, Agent gained
Tolerance for charge estimation: 0.1000
Tolerance between mass estimations: 20.000

TABLE 4

| | | | Calculated | Hypermass |
|---|---|---|---|---|
| Peak | Intensity | Charge | charge | estimation |
| 757.72 | 125000.00 | 4 | 3.99687 | 3026.85 |
| 1010.22 | 45016000.00 | 3 | 2.99687 | 3027.64 |
| 1514.73 | 23987000.00 | 2 | 2.00039 | 3027.45 |

Estimated final mass: 3027.31
Standard deviation: 0.41

FIG. 6: Final HPLC Elugram of SMPH activated miniCD4 peptide.

About 2 mg/ml

### Inj 5 μl ds 25-45

Sample name: FBX13082-190

### Surface Percentage Ratios:

Sorted by: signal

### Multiplicator: 1.0000

### Dilution: 1.0000

Uses multiplicator and dilution factor with internal standard

### Signal 1: DADI A, Sig=230.4 Ref=off

TABLE 5

| | Retention | | | Surface | | |
|---|---|---|---|---|---|---|
| Peak | time | | Width | area | Height | Surface |
| no. | [min] | Type | [min] | [mAU * s] | [mAU] | area % |
| 1 | 12.674 | BV F | 0.0909 | 6.52587 | 1.07449 | 0.3925 |
| 2 | 12.573 | VV | 0.0714 | 6.31252 | 1.19955 | 0.3797 |
| 3 | 12.832 | VV | 0.0909 | 8.14971 | 1.23625 | 0.4902 |
| 4 | 13.027 | VV F | 0.0859 | 16.22212 | 2.63102 | 0.9758 |
| 5 | 13.212 | VB | 0.2055 | 1625.25330 | 120.40638 | 97.7617 |

| | | |
|---|---|---|
| Totals | 1662.46352 | 126.54769 |

Results obtained with improved integrator.

FIG. 7: Mass spectrum of SMPH activated miniCD4 peptide

Copy of the hypermass calculation for +Q1 MCA (10 scans): from FBX13082-190/InfMSpo/c/03/08/05
Criteria used for hypermass calculation
Agent:, Mass: 1.0079, Charge: 1, Agent gained
Tolerance for charge estimation: 0.1000
Tolerance between mass estimations: 20.000

TABLE 6

| | | | Calculated | Hypermass |
|---|---|---|---|---|
| Peak | Intensity | Charge | charge | estimation |
| 791.29 | 322500.00 | 4 | 4.00218 | 3161.12 |
| 1054.52 | 41316500.00 | 3 | 3.00218 | 3160.54 |
| 1581.43 | 7411500.00 | 2 | 1.99944 | 3160.84 |

Estimated final mass: 3160.83
Standard deviation: 0.29

FIG. 8: Diagram for synthesis of maleimide activated miniCD4 peptide via PEO2 linker

FIG. 9: miniCD4 and gp120 interaction

Affinity of synthesized miniCD4 for gp120 was evaluated by Biacore. The results confirm that miniCD4, “designated” by the inventors, with a single lysine is a functional analogue of CD4 protein.

FIG. 10:

HPLC chromatography of the maleimide derivative, mini-CD4-PEO₂, proving that the latter is obtained with a final purity of 77% as described in example V of the description.

FIG. 11:

Mass spectrum showing the mass of the derivative obtained (3205.3938).

### EXAMPLES

### Example I

### Synthesis Diagrams

I.1 Synthesis diagram for the coupling method between a miniCD4 peptide and a polyanion mentioned in application WO 03/089000 (Najjam S. et al., Cytokine 1997, 9 (12): 1013-1022).
Coupling of an amine group to the reducing end of a sugar

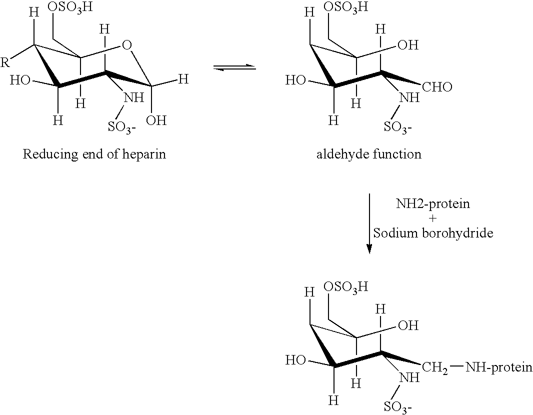

I.2. I.1 Synthesis diagram for the coupling method of the invention

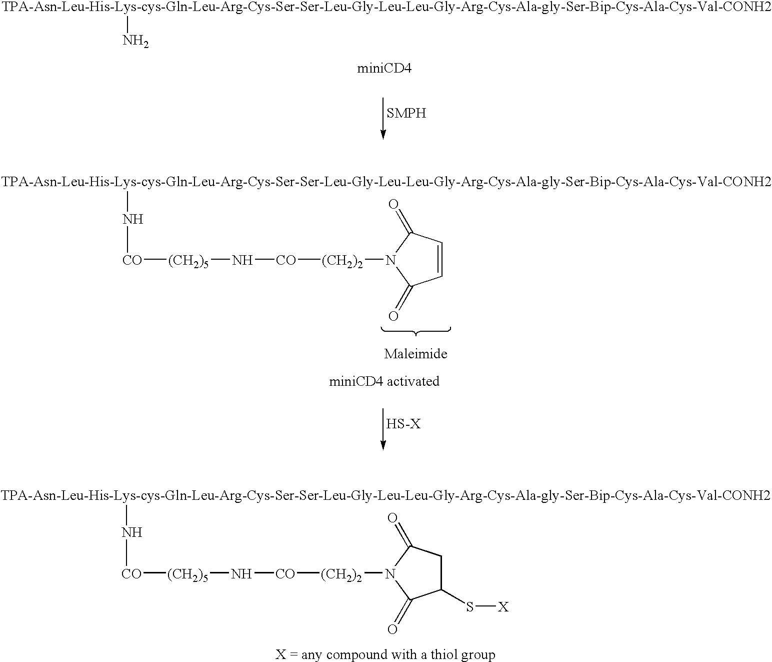

The miniCD4 activation method via incorporation of the maleimide group allows coupling of any compound with a free thiol group (SH) or masked, thiol group (thioacetyl for example). This activated miniCD4 also makes it possible to obtain miniCD4-heparin covalent conjugated molecules insofar as heparin (or any other polysaccharide) will have previously been derivatised by a thiol group.

### Example II

### Chemical Synthesis of Activated MiniCD4 with SMPH or SATP

II.1 Synthesis of miniCD4

A mini-peptide CD4 was synthesized in accordance with the methodology for Fmoc solid phase peptide synthesis (“Fmoc solid phase peptide synthesis, a practical approach”, edited by W. C. Chan and P. D. White, Oxford University Press, 2000) using an Applied Biosystems 433 peptide synthesizer. Starting with 0.1 mmole of amide-Fmoc resin, elongation in stages of the peptide chain was carried out by coupling 10 amino acid equivalents protected by Fmoc and activated by a HATU/DIEA mixture. The N-terminus thiopropionyl group was introduced by SPDP coupling (1.6 equivalent in DMF) on peptide-resin.

After cleaving by TFA/H₂O/EDT/TIS (94/2.5/2.5/1), the peptide was recovered by precipitation in cold diethyl ether. After freeze drying, the raw peptide (156 mg) was reduced overnight in DTT in a 20% acetic acid solution and purified by reverse phase MPLC on a Nucleoprep C18 column, 20 μm, 100 Å (26×313 mm) using a linear gradient 30 to 90% from B to A, over a period of 60 minutes, at a flow rate of 20 ml/min (B=80% CH₃CN/20% aqueous TFA at 0.08%; A=100% aqueous TFA at 0.08%). The pure fractions were collected and freeze dried. The peptide was then folded back using overnight GSH/GSSG treatment. The folded back peptide was purified by MPLC using a 20 to 80% gradient over a period of 60 minutes, giving 8.7 mg of mini-CD4. Purity (93.5%) was verified by analytic reverse phase HPLC on a Nucleosil column C18, 5 μm, 300 Å (4.6×150 mm) using a linear gradient 25 to 35% CH₃CN in aqueous TFA at 0.08% over a period of 20 minutes at a flow rate of 1 ml/min (retention time=15.44 minutes).

ES⁺MS: 2896.32±0.23; expected: 2896.49; yield: 5.5%

II.2 Mini-CD4 Activated with SMPH

The maleimide group was introduced onto the lateral Lys chain of mini-CD4 by reacting 4 SMPH equivalents in phosphate buffer pH=8. The reaction was controlled by HPLC. 100% coupling is achieved after 15 minutes. After purification in a semi preparative Nucleosil C18 column for reverse phase HPLC, 5 μm, 300 Å (10×250 mm) using a linear gradient 25 to 45% CH₃CN in aqueous TFA at 0.08%, over a period of 20 minutes, at a flow rate of 6 ml/min, final purity (97.7%) of mini-CD4 activated with SMPH was controlled by analytic RP-HPLC using a linear gradient 25 to 45% (retention time=13.21 minutes).

ES⁺MS: 3160.83±0.29; expected: 3160.78; yield: 1.9 mg (67%).

II.3 Mini-CD4 Activated with SATP

The thioacetyl group was introduced onto the lateral Lys chain of mini-CD4 by reacting 1 SATP equivalent in phosphate buffer pH=8. The reaction was controlled by HPLC. 46% coupling is achieved after 3 minutes. The mini-CD4 activated by SATP was isolated on a Nucleosil C18 column for semi preparative reverse phase HPLC, 5 μm, 300 Å (10×250 mm) using a linear gradient 20 to 40% CH₃CN in aqueous TFA at 0.08% over a period of 20 minutes, at a flow rate of 6 ml/min. Final purity (100%) of mini-CD4 activated with SATP was controlled by analytic RP-HPLC using a linear gradient 25 to 45% (retention time=13.88 minutes).

ES⁺MS: 3027.31±0.41; expected: 3027.66. This coupling reaction was carried out once and could be optimized by adding directly 2 equivalents of SATP.

### Example III

### Chemical Synthesis of Mini-CD4-GPR1 Peptide

### III.1 Synthesis of GPR1

A GPR1 peptide was synthesized using conventional Fmoc solid phase peptide synthesis, as described for mini-CD4. A Cys residue was incorporated into the C-terminus of the GPR1 sequence to allow specific coupling of the maleimide function of mini-CD4 activated with SMPH. Final purity (91%) of GPR1 peptide was controlled by analytic RP-HPLC using a linear gradient 20 to 40% (retention time=4.64 minutes).

ES⁺MS: 3376.44±0.49; expected: 3376.58. Yield: 12.9 mg (5.8%).

III.2 Coupling of GPR1 to Mini-CD4 Activated with SMPH

A GPR1 peptide (1.5 mg; 0.4 μmole) in 200 μl of phosphate buffer pH=7.4 was added to 200 μl of an aqueous solution of mini-CD4 activated with SMPH (0.95 mg; 0.3 μmole). The reaction was controlled by HPLC. After 15 minutes, the peak corresponding to mini-CD4 activated with SMPH disappeared completely. The GPR1-mini-CD4 peptide candidate was isolated on a Nucleosil C18 column for semi preparative reverse phase HPLC, 5 μm, 300 Å (10×250 mm) using a linear gradient 35 to 55% CH₃CN in aqueous TFA at 0.08% over a period of 20 minutes at a flow rate of 6 ml/min.

Final purity (94.2%) of GPR1-mini-CD4 peptide was controlled by analytic RP-HPLC using a linear gradient 35 to 55% (retention time=12.60 minutes).

ES⁺MS: 6537.26±0.54; expected: 6537.38. Yield: 1.9 mg (96%).

### Example IV

### Relevance of Choice of General Sequence (I) Including One and Only One Lysine Residue in a Defined Position

Relevance of the choice of general sequence (I) including one and only one lysine residue in a defined position was validated by synthesis of a miniCD4 peptide derivatised on Lys by (PEO)4-Biotin using EZ-Link-NHS-(PEO)4-Biotin PIERCE reagent (Rockford, Ill.). Introduction of this Biotin derivative in a defined position in general sequence (I) does not modify binding of gp120 to miniCD4 (Biacore measurement carried out).

The various synthesis processes were not optimised. It should be possible to achieve better yields.

### Example V

### Chemical Synthesis of Maleimide Activated Mini-CD4 Via a PEO₂ Linker

### Refer to FIGS. 8, 10 and 11.

mCD4-PEO₂-maleimide differs from the compound mCD4-SMPH in terms of the type of linker. For reasons of solubility, a polyethylene oxide (PEO₂) linker which is more hydrophilic was incorporated between miniCD4 and the maleimide group.
Synthesis: A solution of 10 mg of mCD4 (MW: 2897; 3.4 mmoles) in 1 ml of H₂O was diluted in 1 ml of phosphate buffer 0.1 M pH 8. 4.5 mg of NHS-PEO₂-Maleimide (MW: 325; 13.8 mmoles; 4 equiv) were added to this cloudy solution in 20 μl of DMSO with stirring. After 10 minutes, 85% (HPLC) of the starting materials was converted into maleimide derivative. Because of the low stability of the maleimide group at pH 8, the coupling reaction was directly loaded onto a SepaK C18 column calibrated with 10% CH₃CN in aqueous TFA 0.08%. The maleimide derivative was eluted with 50% CH₃CN. After freeze drying, the compound was then purified on a semi preparative column. Yield: 5.2 mg (48%), final purity: 77%.

ES⁺: 3205.3938 (expected monoisotopic M: 3205.4211), QTOF Micro Waters MaxEnt1.

HPLC conditions:
Analytic: Nucleosil 5C18 300 Å (4.6×150 mm); linear gradient 25 to 45% CH₃CN in 0.08% aqueous TFA in 20 minutes at a flow rate of 1 ml/min. Detection: 230 nm. mCD4 Rt=10.7 minutes; mCD4-PEO2-Mal Rt=12.8 minutes.
Semi preparative: Nucleosil 5C18 300 Å (10×250 mm); linear gradient 25 to 45% CH₃CN in 0.08% aqueous TFA for 20 minutes at a flow rate of 6 ml/min.
Detection: 230 nm. mCD4-PEO2-Mal Rt=11.4.

### ABBREVIATIONS

Fmoc: 9-fluorenylmethyloxycarbonyl
HATU: hexafluorophosphate N-oxide of N[(dimethylamino)-1H-1,2,3-triazolo[4,5-b]pyridin-1-ylmethylene]-N-methylmethanaminium
DIEA: diisopropylethylamine
SPDP: N-succinimidyl-3(2-pyridyldithio)propionate
TFA: trifluoroacetic acid
EDT: ethanedithiol
TIS: triisopropylsilane
DTT: 1,4-dithiothreitol
MPLC: medium pressure liquid chromatography
ES⁺MS: electrospray mass spectrometry, positive mode
GSH: reduced glutathion
GSSG: oxidised glutathion
HPLC: high-performance liquid chromatography
SMPH: succinimidyl-6-[β-maleimidopropionamido]hexanoate

### SATP: N-succinimidyl-S-acetylthioproprionate

GPR1: receptor 1 coupled to a G protein

1. Process for the preparation of an activated peptide derived from the CD4 receptor, said peptide, once activated, being capable of coupling to an organic molecule by means of covalent binding, and wherein said peptide derived from the CD4 receptor comprises the following general sequence (I):
(SEQ ID NO: 5)
Xaa^{f} - P1 - Lys - Cys - P2 - Cys - P3 - Cys -
Xaa^{g} - Xaa^{h} - Xaaⁱ - Xaa^{j} - Cys - Xaa^{k} - Cys -
Xaa^{l} - Xaa^{m}, (I)
in which:
P1 represents 3 to 6 amino acid residues,
P2 represents 2 to 4 amino acid residues,
P3 represents 6 to 10 amino acid residues,
Xaa^{f} represents N-acetylcysteine (Ac-Cys) or thiopropionic acid (TPA),
Xaa^{g} represents Ala or Gln,
Xaa^{h} represents Gly or (D)Asp or Ser,
Xaaⁱ represents Ser or His or Asn,
Xaa^{j} represents biphenylalanine (Bip), phenylalanine or [beta]—naphthylalanine,
Xaa^{k} represents Thr or Ala, and
Xaa^{l} represents Gly, Val or Leu,
Xaa^{m} represents —NH₂ or —OH,
the amino acid residues in P1, P2 and P3 being natural or non-natural, identical or different, said residues of P1, P2 and P3 being all different from the Lys residue and P1, P2 and P3 having a sequence in common or not,
characterized in that the process involves contacting the peptide of general sequence (I) derived from the CD4 receptor with a bifunctional compound carrying two active groups, where at least one of the two active groups is capable of forming a covalent bond with the free amino group (—NH₂) of the amino acid Lys residue present in general sequence (I).

2. Process for the preparation of an activated peptide according to claim 1, wherein the sequence of the peptide derived from the CD4 receptor of general sequence (I) is chosen from the group consisting of sequences SEQ ID No. 1 and SEQ ID No. 2.

3. Process for the preparation of an activated peptide according to claim 1 or 2, wherein the active group capable of forming a covalent bond with the free amine group (—NH₂) of the amino acid Lys residue present in general sequence (I) is the active group N-hydroxysuccinimide ester (NHS).

4. Process for the preparation of an activated peptide according to claim 3, wherein the two active groups of the bifunctional compound are different and wherein one of the two groups is the NHS active group.

5. Process for the preparation of an activated peptide according to claim 4, wherein the bifunctional compound is succinimidyl-6-[beta-maleimidopropionamido]hexanoate (SMPH).

6. Process for the preparation of an activated peptide according to claim 4, wherein the bifunctional compound is chosen from the group consisting of N-succinimidyl-S-acetylthioacetate (SATA) and N-succinimidyl-S-acetylthiopropionate (SATP).

7. Process for the preparation of an activated peptide according to claim 4, wherein the bifunctional compound is NHS-PEOₙ-maleimide, n being comprised between 2 and 24.

8. Process for the preparation of an activated peptide according to claim 7, wherein n=2, the bifunctional compound being succinimidyl-[(N-maleimidopropionamido)-diethyleneglycol]ester.

9. Process for the preparation of an activated peptide according to any one of claims 1 to 8, wherein Xaa^{f} represents TPA, comprising a preliminary step for the preparation of the peptide derived from the CD4 receptor of general sequence (I), wherein the peptide derived from the CD4 receptor of the following general sequence (II):
(SEQ ID NO: 6)
P1 - Lys - Cys - P2 - Cys - P3 - Cys - Xaa^{g} -
Xaa^{h} - Xaaⁱ - Xaa^{j} - Cys - Xaa^{k} - Cys - Xaa^{l} -
Xaa^{m}, (II)
wherein P1 to P3 and Xaa^{g} to Xaa^{m} are as defined in general sequence (I), is contacted with N-succinimidyl-3-(2-pyridyldithio)propionate (SPDP) in order to incorporate TPA at the N-terminus of said peptide derived from the CD4 receptor of general sequence (II).

10. Activated peptide derived from the CD4 receptor comprising general sequence (I) as defined in claim 1, wherein the amino acid Lys residue is covalently bound, advantageously by an amine bond, to an active group capable of coupling to an organic molecule by means of a covalent bond.

11. Activated peptide according to claim 10, wherein the active group is maleimide.

12. Activated peptide according to claim 11, with the following molecular structure:

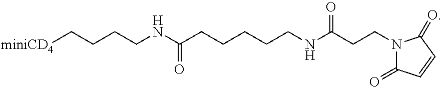

13. Activated peptide according to claim 11, with the following molecular structure:

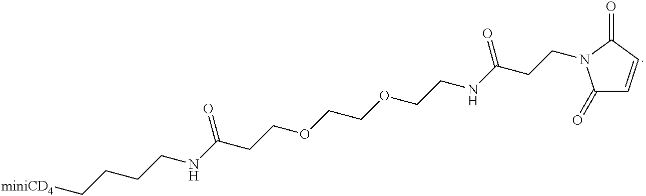

14. Activated peptide according to claim 10, wherein the active group is the thioacetyl group.

15. Activated peptide according to claim 14, with the following molecular structure:

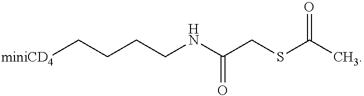

16. Activated peptide according to claim 14, with the following molecular structure:

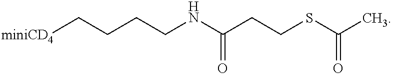

17. Process for the preparation of a conjugated molecule comprising a peptide derived from the CD4 receptor coupled by covalent binding to an organic molecule, said peptide derived from the CD4 receptor comprising general sequence (I) as defined in claim 1,
characterized in that the process involves contacting the activated peptide according to any one of claims 10 to 16 with the organic molecule.

18. Process for the preparation of a conjugated molecule according to claim 17, wherein the activated peptide is the peptide according to one of claims 11 to 13 and the organic molecule carries a thiol or thioacetyl group.

19. Process for the preparation of a conjugated molecule according to claim 18, wherein the organic molecule carrying a thiol group is the peptide GPR1 whose sequence is chosen from among the group consisting of sequences SEQ ID No. 3 and SEQ ID No. 4.

20. Process for the preparation of a conjugated molecule according to claim 18, wherein the organic molecule carrying a thiol group is chosen from the group consisting of peptides essentially carrying acid residues, peptides essentially carrying phosphorylable residues and peptides essentially carrying sulphatable residues.

21. Process for the preparation of a conjugated molecule according to claim 18, wherein the organic molecule carrying a thiol group is a modified polyanion carrying the thiol group.

22. Process for the preparation of a conjugated molecule according to claim 21, wherein the modified polyanion carrying the thiol group is chosen from the group consisting of heparin and heparan sulphate and has a degree of polymerisation dp of 10 to 24.

23. Process for the preparation of a conjugated molecule according to claim 17, wherein the activated peptide is the peptide according to one of claims 14 to 16 and the organic molecule carries a maleimide or halogen group.

24. Conjugated molecule including a peptide derived from the CD4 receptor comprising general sequence (I) as defined in claim 1 coupled to an organic molecule wherein the peptide derived from the CD4 receptor and the organic molecule are coupled to each other with a linker and wherein the amino acid Lys residue of general sequence (I) forms an amino bond with the linker.

25. Conjugated molecule according to claim 24 wherein general sequence (I) is the sequence SEQ ID No. 1 and the organic molecule carrying a thiol group is peptide GPR1 whose sequence is chosen form the group consisting of sequences SEQ ID No. 3 and SEQ ID No. 4.

26. Conjugated molecule according to claim 24, wherein general sequence (I) is the sequence SEQ ID No. 1 and the organic molecule carrying a thiol group is the modified polyanion carrying the thiol group.

27. Use of the activated peptide according to any of claims 11 to 13, for the coupling to an organic molecule having a thiol or thioacetyl group.

28. Use of the activated peptide according to any of claims 14 to 16, for the coupling to an organic molecule carrying a maleimide or halogen group.

29. Use of the activated peptide according to any of claims 10 to 16, for the manufacture of a medicament for antiviral treatment.

30. Use according to claim 29, wherein the medicament is intended to treat AIDS.

31. Conjugated molecule according to claim 25 or 26, for use as medicament.

32. Use of conjugated molecule according to claim 25 or 26, for the manufacture of a medicament for the treatment of AIDS.
